(19) United States
(12) Patent Application Publication
(10) Pub. No.: US 2023/0270989 A1
(43) Pub. Date: Aug. 31, 2023

(54) SINGLE CAVITY HEMOSTASIS VALVE HUB FOR INTRODUCER SHEATHS

(71) Applicants: Boston Scientific Medical Device Limited, Galway (IE); Boston Scientific Scimed Inc., Maple Grove, MN (US)

(72) Inventors: Neeraj Kumar Sharma, New Delhi (IN); Sumit Agrawal, haryana (IN); Rupesh Kumar Pahwa, Gurugram (IN); Qian Liu, Plymouth, MN (US); Swami Upadhyay, (US)

(21) Appl. No.: 18/114,080

(22) Filed: Feb. 24, 2023

Related U.S. Application Data
(60) Provisional application No. 63/314,102, filed on Feb. 25, 2022.

(51) Int. Cl. A61M 39/06; A61M 25/00

(52) U.S. Cl. CPC A61M 39/06; A61M 25/0097; A61M 25/0662

(57) ABSTRACT

A hemostasis valve hub for use with an introducer sheath includes a base having a securing mechanism for securing the hemostasis valve hub to the introducer sheath, an outer shell securable to the base and defining an interior volume, a shutter helix section disposed within the interior volume and including a plurality of fins arranged in a helical fashion, a brush section disposed within the interior volume adjacent the disk shutter section, the brush section including a plurality of brushes arranged in a helical fashion, and a seal positioned within the base and adjacent the brush section, the seal having a partial cross slit arrangement.

### CROSS-REFERENCE TO RELATED APPLICATIONS

This application claims priority to Provisional Application No. 63/314,102, filed Feb. 25, 2022, which is herein incorporated by reference in its entirety.

### TECHNICAL FIELD

The present disclosure relates to a hub for use with introducer sheaths. More specifically, the present disclosure relates to a single cavity hemostasis valve hub for use with large bore introducer sheaths.

### BACKGROUND

In various procedures for delivering intravascular medical devices, an introducer sheath is inserted into a blood vessel of a patient, for example a femoral artery, and medical devices are inserted into the introducer sheath for introduction into the blood vessel. In various instances, the medical devices include catheters or other medical devices such as a blood pump. In various instances, multiple medical devices need to be introduced into or inserted through the blood vessel at the same time. A hemostasis valve hub may be incorporated at a proximal end of the large bore introducer sheath to reduce blood leakage as devices are being inserted, positioned, and removed. There is a need for an optimized hemostasis valve hub that allows for the passage of multiple devices into the introducer sheath while still minimizing blood leakage and facilitating passage of the device through an introducer sheath.

### SUMMARY

In Example 1, a hemostasis valve hub for use with an introducer sheath includes a base having a securing mechanism for securing the hemostasis valve hub to the introducer sheath, an outer shell securable to the base and defining an interior volume, a shutter helix section disposed within the interior volume and including a plurality of fins arranged in a helical fashion, a brush section disposed within the interior volume adjacent the shutter helix section, the brush section including a plurality of brushes arranged in a helical fashion, and a seal positioned within the base, the seal having a partial cross slit arrangement.

In Example 2, the hemostasis valve hub of Example 1 includes wherein the securing mechanism is a ferrule positioned within the base for engaging with the introduce sheath and the hemostasis valve hub.

In Example 3, the hemostasis valve hub of Example 1 or Example 2 includes wherein the outer shell comprises a top cap and a bottom cap coupled with the top cap.

In Example 4, the hemostasis valve hub of Example 3 includes wherein the seal is positioned adjacent the top cap and the brush section.

In Example 5, the hemostasis valve hub of any one of Examples 1-4 includes wherein the seal is configured to have a diameter of at least 8.0 mm.

In Example 6, the hemostasis valve hub of any one of Examples 1-5 includes wherein the base comprises a plurality of suture rings for securing the position of the device.

In Example 7, the hemostasis valve hub of Example 6 includes wherein the base, the outer shell, the seal assembly and the hub cap are aligned longitudinally to define a lumen.

In Example 8, a delivery system for inserting a plurality of medical devices into a blood vessel includes an introducer sheath having a proximal end and a distal end and a hemostasis valve hub configured for engaging the proximal end of the introducer sheath. The hemostasis valve hub includes a base having a securing mechanism for securing the hemostasis valve hub to the introducer sheath, an outer shell securable to the base and defining an interior volume, a shutter helix section disposed within the interior volume and including a plurality of fins arranged in a helical fashion, a brush section disposed within the interior volume adjacent the shutter helix section, the brush section including a plurality of brushes arranged in a helical fashion, and a seal positioned within the base and adjacent the brush section, the seal having a partial cross slit arrangement.

In Example 9, the delivery system of Example 8 includes wherein the outer shell comprises a top cap coupled to a bottom cap.

In Example 10, the delivery system of Example 9 includes wherein the seal is positioned adjacent the top cap and the brush section.

In Example 11, the delivery system of Example 10 includes wherein the bottom cap comprises an O-ring seal for forming a seal between the bottom cap and the base.

In Example 12, the delivery system of any one of Examples 8-11 includes wherein the securing mechanism is a ferrule positioned within the base of the hub for engaging the base and the introducer sheath.

In Example 13, a method of delivering at least one medical device into the blood vessel includes securing a hub base to a proximal end of an introducer sheath with a securing mechanism, securing an outer shell containing at least a seal, shutter helix section, and a brush section to the hub base and securing a hub cap to the hub base, inserting the introducer sheath into the blood vessel, and inserting at least one medical device into a lumen defined by the hub cap, through the outer shell and through the hub base to extend the medical device into the introducer sheath.

In Example 14, the method of Example 13 includes wherein the brush section comprises a plurality of brushes positioned in a helical arrangement, such that upon insertion of the medical device, the plurality of brushes sealingly engage with the medical device.

In Example 15, the method of Example 13 or Example 14 includes wherein the securing mechanism for securing the hub base to the introducer sheath includes a ferrule positioned within the hub base.

In Example 16, a hemostasis valve hub for use with an introducer sheath includes a base having a securing mechanism for securing the hemostasis valve hub to the introducer sheath, an outer shell securable to the base and defining an interior volume, a shutter helix section disposed within the interior volume and including a plurality of fins arranged in a helical fashion, a brush section disposed within the interior volume adjacent the shutter helix section, the brush section including a plurality of brushes arranged in a helical fashion, and a seal positioned within the base and adjacent the brush section, the seal having a partial cross slit arrangement.

In Example 17, the hemostasis valve hub of Example 16 includes wherein the securing mechanism is a ferrule positioned within the base for engaging with the introducer sheath and the hemostasis valve hub.

In Example 18, the hemostasis valve hub of Example 16 includes wherein the outer shell comprises a top cap and a bottom cap coupled with the top cap.

In Example 19, the hemostasis valve hub of Example 18 includes wherein the seal is positioned adjacent the top cap and the brush section.

In Example 20, the hemostasis valve hub of Example 16 includes wherein the seal is configured to have a diameter of at least 8.0 mm.

In Example 21, the hemostasis valve hub of Example 20 includes wherein the seal is composed of silicone.

In Example 22, the hemostasis valve hub of Example 16 includes wherein the shutter helix section comprises a plurality of fins extending radially inward from an outer ring.

In Example 23, the hemostasis valve hub of Example 16, includes wherein the base comprises a plurality of suture rings for securing the position of the device.

In Example 24, the hemostasis valve hub of Example 16, includes wherein the hub comprises a hub cap configured for engaging with the outer shell.

In Example 25, the hemostasis valve hub of Example 24 includes wherein the base, the outer shell, the seal assembly and the hub cap are aligned longitudinally to define a lumen.

In Example 26, a delivery system for inserting a plurality of medical devices into a blood vessel includes an introducer sheath having a proximal end and a distal end and a hemostasis valve hub configured for engaging the proximal end of the introducer sheath. The hemostasis valve hub includes a base having a securing mechanism for securing the hemostasis valve hub to the introducer sheath, an outer shell securable to the base and defining an interior volume, a shutter helix section disposed within the interior volume and including a plurality of fins arranged in a helical fashion, a brush section disposed within the interior volume adjacent the shutter helix section, the brush section including a plurality of brushes arranged in a helical fashion, and a seal positioned within the base and adjacent the brush section, the seal having a partial cross slit arrangement.

In Example 27, the delivery system of Example 26 includes wherein the outer shell comprises a top cap coupled to a bottom cap.

In Example 28, the delivery system of Example 27 includes wherein the seal is positioned adjacent the top cap and the brush section.

In Example 29, the delivery system of Example 26 includes wherein the bottom cap comprises an O-ring seal for forming a seal between the bottom cap and the base.

In Example 30, the delivery system of Example 26 includes wherein the securing mechanism is a ferrule positioned within the base of the hub for engaging the base and the introducer sheath.

In Example 31, a method of delivering at least one medical device into the blood vessel includes securing a hub base to a proximal end of an introducer sheath with a securing mechanism, securing an outer shell containing at least a seal, a shutter helix section, and a brush section to the hub base and securing a hub cap to the hub base, inserting the introducer sheath into the blood vessel, and inserting at least one medical device into a lumen defined by the hub cap, through the outer shell and through the hub base to extend the medical device into the introducer sheath.

In Example 32, the method of Example 31 further includes wherein the method further includes inserting a second medical device through the lumen.

In Example 33, the method of Example 31 further includes wherein the brush section comprises a plurality of brushes positioned in a helical arrangement, such that upon insertion of the medical device, the plurality of brushes sealingly engage with the medical device.

In Example 34, the method of Example 31 further includes wherein the securing mechanism for securing the hub base to the introducer sheath includes a ferrule positioned within the hub base.

In Example 35, the method of Example 31 further includes wherein the securing mechanism for securing the hub base to the introducer sheath is a threaded cap for engaging with the proximal end of the introducer sheath and the hub base.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a cross sectional view of an introducer sheath after insertion into a blood vessel, in accordance with embodiments of the present disclosure.

FIG. 2 illustrates a cross sectional view of the introducer sheath after insertion into a blood vessel and insertion of a medical device into the introducer sheath, in accordance with embodiments of the present disclosure.

FIG. 3 is a side view of a hemostasis valve hub for use with an introducer sheath, in accordance with embodiments of the present disclosure.

FIG. 4 is an exploded view of the hemostasis valve hub of FIG. 3, in accordance with embodiments of the present disclosure.

FIG. 5 is a cross sectional view of the hemostasis valve hub of FIG. 3, in accordance with embodiments of the present disclosure.

FIG. 6 is an exploded view of a portion of the hemostasis valve hub of FIG. 5, in accordance with embodiments of the present disclosure.

FIG. 7 is an enlarged view of a shell cap of the hemostasis valve hub, in accordance with embodiments of the present disclosure.

FIG. 8 is an enlarged view of a seal of the hemostasis valve hub, in accordance with embodiments of the present disclosure.

FIG. 9A is an enlarged view of a brush section of the hemostasis valve hub, in accordance with embodiments of the present disclosure.

FIG. 9B is an enlarged view of a brush of the brush section of FIG. 9A, in accordance with embodiments of the present disclosure.

FIG. 10 illustrates an enlarged view of a shutter helix for use with the hemostasis valve hub, in accordance with embodiments of the present disclosure.

FIG. 11 illustrates an enlarged view of a shell cap for use with the hemostasis valve hub, in accordance with embodiments of the present disclosure.

FIG. 12 illustrates an exploded view of a hemostasis valve hub, in accordance with embodiments of the present disclosure.

FIG. 13 illustrates a flow chart for a method of inserting a medical device into a blood vessel, in accordance with embodiments of the present disclosure.

### DETAILED DESCRIPTION

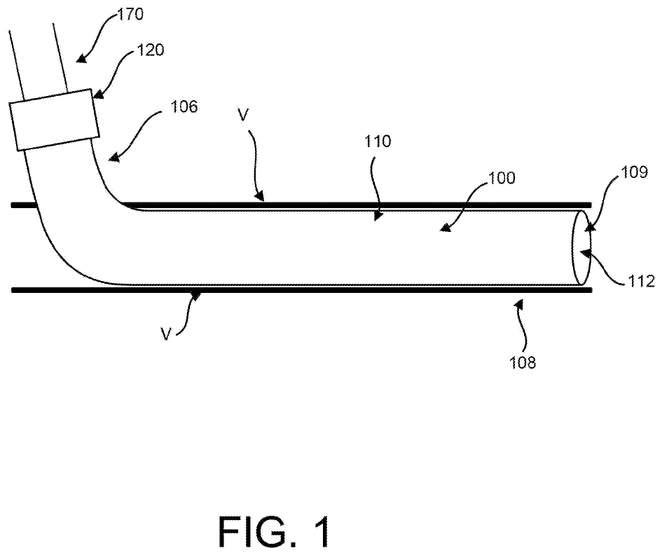

FIG. 1 illustrates a side cross sectional view of a blood vessel V with an introducer sheath 100, inserted at least partially into the blood vessel V. In some embodiments, the introducer sheath 100 is used for facilitating the passage of various relatively large medical devices, such as a blood pump as will be described further herein, through the introducer sheath 100 and into the blood vessel V. Hence, the introducer sheath 100 may be referred to as a large bore introducer sheath. The introducer sheath 100 comprises a proximal end 106 and a distal end 108 that is opposite the proximal end 106. In embodiments, the proximal end 106 is a flared proximal end. The introducer sheath 100 includes a proximal opening (not shown) adjacent the proximal end 106 and a distal opening 109 adjacent the distal end 108. A body portion 110 of the introducer sheath 100 extends between the proximal end 106 and the distal end 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or any other applicable polymer. A hub 120 is commonly included at the proximal opening. The hub 120, also referred to herein as a hemostasis valve hub, is configured for hemostasis, i.e, to prevent blood from leaking out of the introducer sheath during use. In various instances, it may be desired for hub 120 to be configured for the passage of multiple medical devices through the introducer sheath 100 at one time. In these embodiments, it is beneficial to ensure that the hub 120 provides sufficient sealing between the multiple medical devices, for example catheters, such that there is reduced blood leakage from the introducer sheath 100 during insertion, operation, and removing of devices. The use of a hub inserted at the proximal end 106 of introducer sheath 100 provides an additional method of sealing the introducer sheath 100. An example embodiment of the hub 120 will be described further herein. As shown in FIG. 1, the hub 120 may be used to receive a catheter 170. The catheter 170 extends through the hub 120 and the introducer sheath 100 and may be coupled to a proximal end of a blood pump which may be inserted into the introducer sheath 100, as will be described further herein.

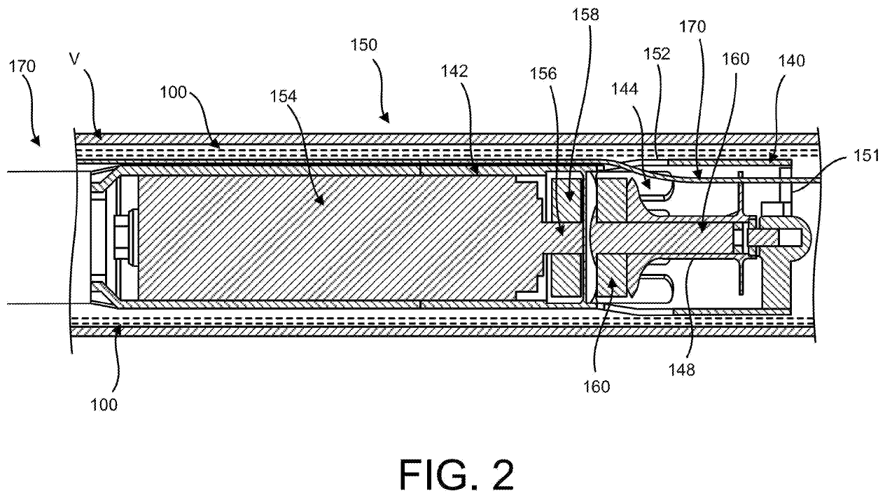

FIG. 2 illustrates a cross-sectional view of the introducer sheath 100 of FIG. 1 after insertion of a medical device, illustratively a blood pump 150, into the introducer sheath 100. The blood pump 150 generally includes an impeller assembly housing 140 and a motor housing 142. In some embodiments, the impeller assembly housing 140 and the motor housing 142 may be integrally or monolithically constructed. In other embodiments, the impeller assembly housing 140 and the motor housing 142 may be separate components configured to be removably or permanently coupled.

The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrated, and in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula (not shown), and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. Additionally, as illustrated in FIG. 2, the catheter 170 extends from a proximal end of the blood pump 150. In some embodiments, the catheter 170 may be coupled to the motor housing 142 through a tapering connector and/or various other connecting means. The catheter 170 may have a flexible construction to facilitate to the delivery of the blood pump 150. While the introducer sheath 100 is illustrated above with the use of the blood pump 150, various other medical devices may be used in conjunction with the introducer sheath 100 and the hemostasis valve hub 120, and the blood pump 150 is provided as an example. For example, another variation of a blood pump may be used in conjunction with the introducer sheath 100. In other examples, a medical device other than a blood pump may be incorporated.

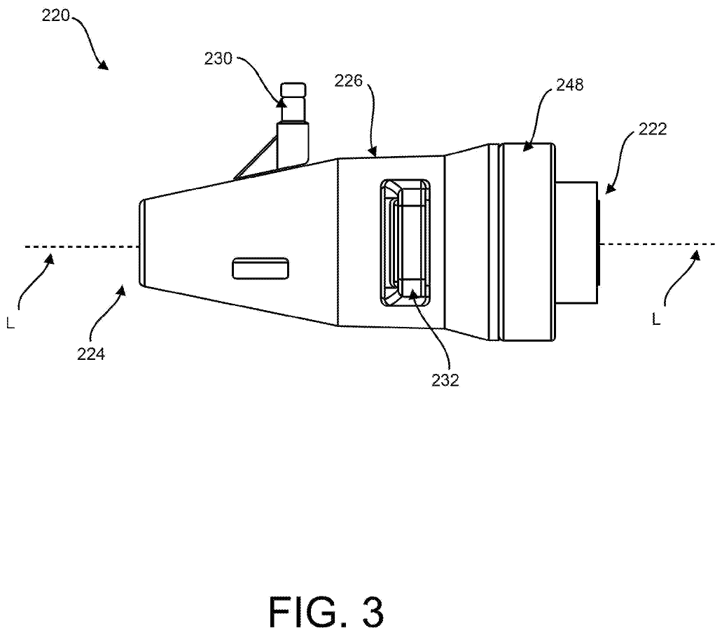

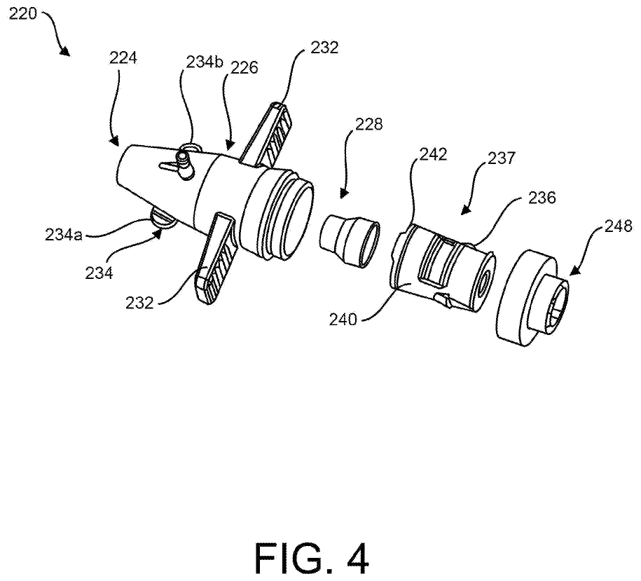

FIGS. 3 and 4 illustrate a hemostasis valve hub 220 that may be used for receiving a medical device, for example catheter 170 coupled with blood pump 150 (FIG. 2), for insertion into the introducer sheath 100 (FIG. 1) and the blood vessel V (FIG. 1). Specifically, the hemostasis valve hub 220 has a proximal end 222, a distal end 224, and a longitudinal axis L extending between the proximal end 222 and the distal end 224. The hemostasis valve hub 220 includes a hub base 226 at the distal end 224. As illustrated, the hemostasis valve hub 220 also includes an access port 230 extending from the hub base 226. The access port 230 may be used for receiving additional fluids and/or medical devices for irrigation within the hemostasis valve hub 220 or the introducer sheath 100. The hub base 226 additionally includes at least one flange 232 that may be used by a physician for grasping the hemostasis valve hub 220 when securing the hemostasis valve hub 220 onto the introducer sheath 100 (FIG. 1). In some embodiments, the hemostasis valve hub 220 comprises two or more flanges 232. The hemostasis valve hub 220 may also include one or more suture rings 234. For example, as shown in FIG. 4, the one or more suture rings 234 comprises a first suture ring 234a and a second suture ring 234b. The suture rings 234 may be configured for receiving a suture to secure the positioning of the hemostasis valve hub 220.

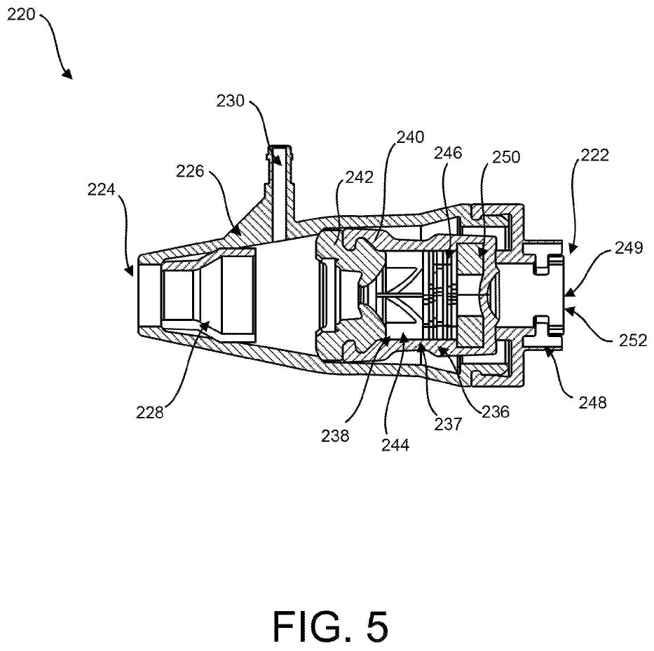

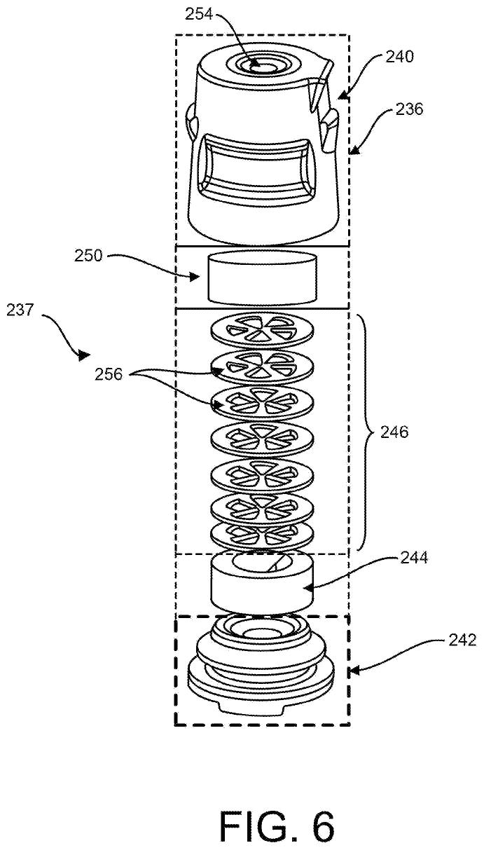

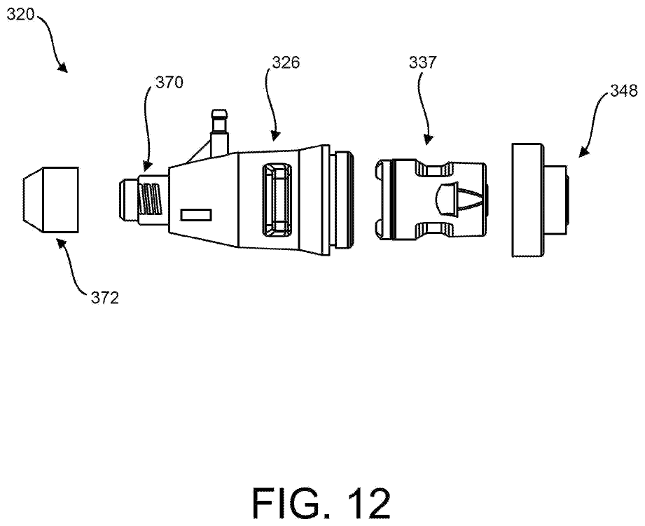

FIG. 5 illustrates a cross sectional view of the hemostasis valve hub 220 as shown in FIGS. 3-4. As shown, the hub base 226 comprises a securing mechanism at the distal end 224 of the hemostasis valve hub 220 for coupling the hemostasis valve hub 220 to the proximal end 106 (FIG. 1) of the introducer sheath 100 (FIG. 1). More specifically, in the illustrative embodiment of FIG. 5, the hub base 226 comprises a ferrule 228 for insertion into the hub base 226 and configured for engagement with the proximal end 106 of the introducer sheath 100. However, various other securing mechanisms may be used, for example as will be described with reference to FIG. 12. The hub base 226 additionally comprises an outer shell 236 enclosing an interior region 238 that includes a seal assembly 237. More specifically, the outer shell 236 comprises a top cap 240 engaged with a bottom cap 242 to define the interior region 238. The seal assembly 237 comprises a shutter helix 244, a brush section 246 and a seal 250 within the outer shell 236. The components of the seal assembly 237 will be described further herein with reference to FIGS. 6-11. Further, as illustrated in FIG. 5, the hub base 226 and the top cap 240 of the outer shell 236 are engaged with a hub cap 248. The hub cap 248 comprises an opening 252, for receiving a medical device, for example a catheter. The opening 252 extends into a lumen 249 that extends through the hemostasis valve hub 220. In some embodiments, the hub cap 248 may be configured to couple to a tightening port (not shown) used to secure a medical device, such as a catheter, passing through the hub 220.

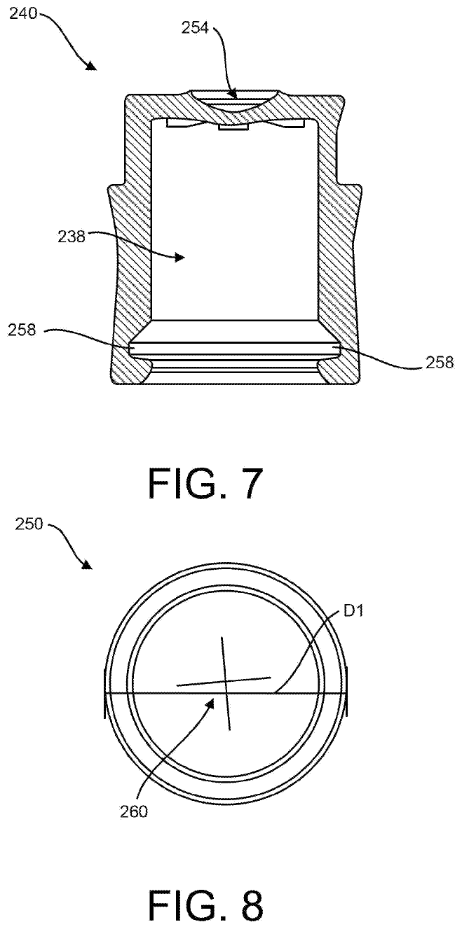

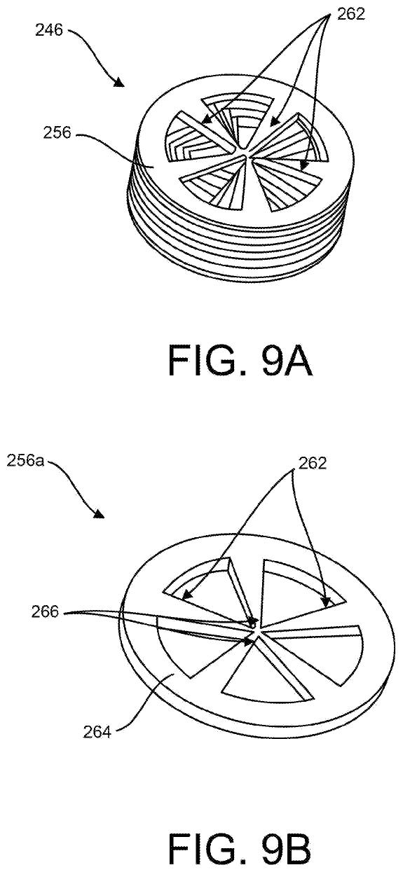

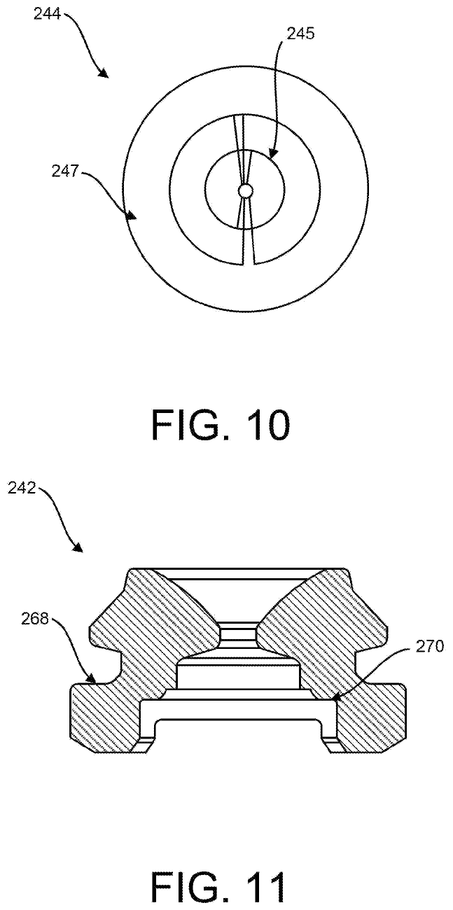

With reference now to FIG. 6, the outer shell 236 and the seal assembly 237 will be described further. As illustrated, the top cap 240 has a generally cylindrical shape with an opening 254 extending through the top cap 240 and configured for alignment with the opening 252 of the hub cap 248. Positioned below the top cap 240 and configured for reception within the top cap 240 is the seal 250. The seal 250 may be a cylindrical seal with a partial cross slit within the center of the seal 250, as will be described further with reference FIG. 8. Further, the brush section 246 is positioned below the seal 250. As will be described further with reference to FIGS. 9A-9B, the brush section 246 comprises a plurality of brushes 256, or brush discs, stacked adjacent one another. The brush section 246 is configured for providing a sealing engagement with the medical device. As illustrated, positioned below the brush section 246 is the shutter helix 244. The shutter helix 244 is configured for providing sealed engagement between the hemostasis valve hub 220 and the various medical devices, as will be described further with reference to FIG. 10 Further, below the shutter helix 244 is the bottom cap 242 of the outer shell 236. The above components of the seal assembly 237 and outer shell 236 will be described in further detail herein with reference FIGS. 7-11.

FIG. 7 illustrates a cross section of the top cap 240 of the outer shell 236 and a portion of the interior region 238 defined within the top cap 240. As illustrated, the top cap 240 comprises the opening 254 at the topmost surface of the top cap 240. The top cap 240 may include engaging features 258 that are configured for engagement with the hub cap 248. While illustrated as generally cylindrically shaped, the top cap 240 may have varying configurations. Further, the top cap 240 may be composed of materials like polymer, thermoset, rubber or thermoset elastomer (TSE), or silicone rubber.

FIG. 8 illustrates the seal 250 for use within the seal assembly 237 (FIG. 6). The seal 250 is generally cylindrical in shape and has a circular cross section with partial cross slits 260 arranged within a center of the seal 250. As previously described, the partial cross slits 260 are configured such that the at least one medical device inserted through the opening of the top cap 240 (FIG. 7) is then received within the partial cross slits 260 of the seal 250, such that the catheter sealingly engages with the medical device. The seal 250 is composed of silicone, however various other materials may be used. For example, materials like polymer, thermoset, rubber or thermoset elastomer (TSE), or silicone rubber may be used. The seal 250 may have a diameter D1 that has a value between 8 mm and 12 mm. The configuration of the seal 250 may provide the additional hemostasis advantage for longer duration of usage. The partial cross slit seal 250 grips the device passed through without absorbing the fluid/blood that may be passing though the device. The configuration of the seal 250 allows the hemostasis of blood for longer duration during usage of the introducer sheath 100 in the blood vessel V.

As previously described, adjacent the seal 250 is the brush section 246. FIG. 9A illustrates the brush section 246 composed of plurality of brushes 256 stacked upon one another. As illustrated in both FIGS. 9A and 9B, each brush, for example a brush 256a, comprises a plurality of bristles 262 extending radially inward from an outer circumference 264 of the brush 256a. Specifically, each bristle 262 has a generally triangular shape and an apex 266 at the most radially inward point of each bristle 262. When the plurality of brushes 256 are stacked with one another, the bristles 262 are offset helically to form a fan like arrangement. When a medical device is inserted through the top cap 240 and the seal 250, the medical device, for example a catheter, extends through the brush section 246. Upon insertion of the catheter through the brush section 246, the catheter extends through the brush section 246 at a radial center point of the brush section 246. The radial center point is defined as the position wherein the apices 266 of each bristle 262 converge with one another. In certain instances, when the catheter is inserted through the brush section 246, a channel of the catheter is engaged by various of the apices 266 of the brush section 246. This configuration increases the sealing between the catheter, the introducer sheath 100 and the hemostasis valve hub 220, and additionally contributes to a stabilized axial position of the catheter within the hemostasis valve hub 220. Additionally, when more than one medical device is inserted through the brush section 246, the configuration of the bristles 262 ensures that each of the multiple medical devices, or catheters, are sealing engaged by the brush section 246. Further, when multiple medical devices are inserted into the hemostasis valve hub 220, the brush section 246 configuration may stabilize the axial position of each medical device.

FIG. 10 illustrates a top cross-sectional view of the shutter helix 244. The shutter helix 244 comprises a plurality of fins 245 that extend radially inward from an outer ring 247 of the shutter helix 244. Similar to as described with reference to the brush section 246, when a medical device is inserted into the hemostasis valve hub 220, for example a catheter, the catheter extends through a general center of the shutter helix 244. The plurality of fins 245 may deform as the catheter is inserted through the shutter helix 244, however, the plurality of fins 245 are biased into contact with the catheter. In this way, as the catheter is inserted through the shutter helix 244, the plurality of fins 245 work to maintain a hemostatic seal between the catheter, the hemostasis valve hub 220 and the introducer sheath 100 (FIG. 1). In some embodiments, a second catheter and/or medical device may be inserted through the hemostasis valve hub 220 as well. In these embodiments, the plurality of fins 245 are configured such that the shutter helix 244 maintains a hemostatic valve around the second catheter, as well. In various embodiments, the shutter helix 244 has a thickness that ranges between 2 mm to 4 mm, and the plurality of fins 245 may include at least two fins of thickness 1 mm with tapered down profile.

FIG. 11 illustrates a cross sectional view of the bottom cap 242 of the outer shell 236. As previously described, the bottom cap 242 is configured for engagement with the top cap 240 to form the outer shell 236. More specifically, as illustrated, the bottom cap 242 comprises engagement features 268 for coupling with the top cap 240. The bottom cap 242 additionally comprises a circumferential groove 270 for receiving a seal (not shown), for example an O-ring seal, configured for being received in the circumferential groove 270 of the bottom cap 242. The O-ring seal may contribute to a fluid tight seal between the bottom cap 242 when the bottom cap 242 is engaged with the hub base 226. In this way, the above-described features can be engaged with one another to form the seal assembly 237 positioned within the outer shell 236. Any of the above-described features may be altered in configuration, removed, or added to the seal assembly 237 in order to alter the sealing ability of the seal assembly 237. For example, the number of brushes 256 of the brush section 246 may be increased and/or reduced and the seal 250 may be increased or decreased in diameter or thickness. As previously described, the seal assembly 237 and the outer shell 236 are positioned between the hub base 226 and the hub cap 248. As such, when a medical device, such as the catheter, is inserted through the hub cap 248, the catheter extends through the lumen 249 (FIG. 5) and as such, through the seal assembly 237 and through the hub base 226 of the hemostasis valve hub 220. The catheter can then extend out of the distal end 224 of the hemostasis valve hub 220 and into the introducer sheath 100. Further, in various embodiments, an additional medical device (i.e., a second catheter) can be inserted through the hemostasis valve hub 220, through the lumen 249, and into the introducer sheath 100.

FIG. 12 illustrates an additional embodiment of a hemostasis valve hub 320, which may be similar to or the same as the hemostasis valve hub 220 with the exception of the securing mechanism and a distal end of the hub base. Specifically, FIG. 12 illustrates the hemostasis valve hub 320 comprising the hub base 326 engaged with a seal assembly 337 that engages with a hub cap 348. The hub base 326, the seal assembly 337, and a hub cap 348 may be the same as the hub base 226, the seal assembly 237, and the hub cap 248 as described with reference to FIG. 4. However, as illustrated, the hub base 326 comprises a narrowed portion 370 towards a distal end 324 of the hemostasis valve hub 320. The narrowed portion 370 is configured for engagement with a threaded cap 372. In this way, the threaded cap 372 engages with both the hub base 326 and the proximal end 106 (FIG. 1) of the introducer sheath 100 (FIG. 1) to secure the hemostasis valve hub 320 to the introducer sheath 100.

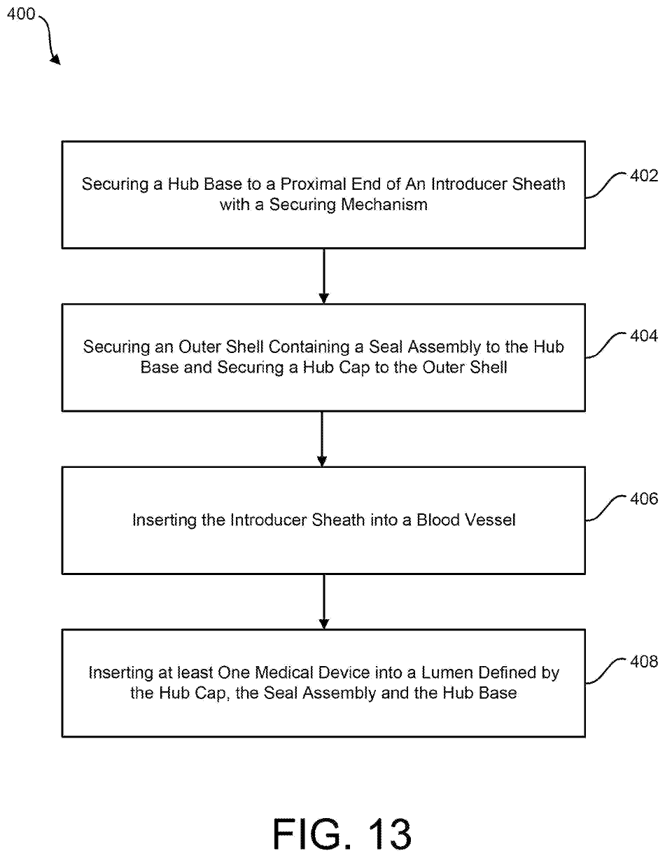

With reference to FIG. 13 and FIG. 1, a method 400 for delivering at least one medical device into the blood vessel V with using the hemostasis valve hub 220 as described with reference to FIGS. 3-10, will be described. At block 402, the method 400 first includes securing the hub base 226 to the proximal end 106 of the introducer sheath 100 with a securing mechanism. In various embodiments, the securing mechanism includes placing the ferrule 228 into the hub base 226 to secure the hub base 226 with the proximal end 106 of the introducer sheath 100. In various other embodiments, the method 400 may be applied with use of the hemostasis valve hub 320 as described with reference to FIG. 12, and the securing mechanism may comprise the threaded cap 372 in engagement with the narrowed portion 370.

At block 404, the method 400 further includes securing the outer shell 236 containing the seal assembly 237 to the hub base 226 and securing the hub cap 248 to the outer shell 236. More specifically, the bottom cap 242 is secured to the hub base 226 and the top cap 240 is secured to the hub cap 248. At block 406, the method 400 further includes inserting the introducer sheath 100 into the blood vessel V.

Further, at block 408, the method 400 further includes inserting at least one medical device into the lumen 249 that is defined by the hub cap 248, the seal assembly 237, and the hub base 226. Inserting the at least one medical device may include inserting a catheter, for example the catheter 170 (FIG. 1) through the hemostasis valve hub 220 and extending the catheter 170 out of the distal end 224 of the hemostasis valve hub 220. As previously described, the insertion of the catheter 170 through the seal assembly 237 causes sealing engagement between the catheter 170 and the hemostasis valve hub 220. In some embodiments, an additional medical device, for example a second catheter, may be inserted into the hemostasis valve hub 220, as well. As a result of the configuration of the outer shell 236 and the various elements of the seal assembly 237, the at least two medical devices can be inserted through the hemostasis valve hub 220 at the same time. In this way, the hemostasis valve hub 220 works to provide a single cavity hemostasis valve hub for receiving a plurality of medical devices through the hemostasis valve hub 220 and into the introducer sheath 100 with reduced leakage and increased axial stability of the medical devices.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A hemostasis valve hub for use with an introducer sheath, comprising:
a base having a securing mechanism for securing the hemostasis valve hub to the introducer sheath;
an outer shell securable to the base and defining an interior volume;
a shutter helix section disposed within the interior volume and including a plurality of fins arranged in a helical fashion;
a brush section disposed within the interior volume adjacent the shutter helix section, the brush section including a plurality of brushes arranged in a helical fashion; and
a seal positioned within the base and adjacent the brush section, the seal having a partial cross slit arrangement.

2. The hemostasis valve hub of claim 1, wherein the securing mechanism is a ferrule positioned within the base for engaging with the introducer sheath and the hemostasis valve hub.

3. The hemostasis valve hub of claim 1, wherein the outer shell comprises a top cap and a bottom cap coupled with the top cap.

4. The hemostasis valve hub of claim 3, wherein the seal is positioned adjacent the top cap and the brush section.

5. The hemostasis valve hub of claim 1, wherein the seal is configured to have a diameter of at least 8.0 mm.

6. The hemostasis valve hub of claim 5, wherein the seal is composed of silicone.

7. The hemostasis valve hub of claim 1, wherein the shutter helix section comprises a plurality of fins extending radially inward from an outer ring.

8. The hemostasis valve hub of claim 1, wherein the base comprises a plurality of suture rings for securing the position of the device.

9. The hemostasis valve hub of claim 1, wherein the hub comprises a hub cap configured for engaging with the outer shell.

10. The hemostasis valve hub of claim 9, wherein the base, the outer shell, the seal assembly and the hub cap are aligned longitudinally to define a lumen.

11. A delivery system for inserting a plurality of medical devices into a blood vessel, comprising:
an introducer sheath having a proximal end and a distal end; and
a hemostasis valve hub configured for engaging the proximal end of the introducer sheath, the hemostasis valve hub comprising:
a base having a securing mechanism for securing the hemostasis valve hub to the introducer sheath;
an outer shell securable to the base and defining an interior volume;
a shutter helix section disposed within the interior volume and including a plurality of fins arranged in a helical fashion;
a brush section disposed within the interior volume adjacent the shutter helix section, the brush section including a plurality of brushes arranged in a helical fashion; and
a seal positioned within the base and adjacent the brush section, the seal having a partial cross slit arrangement.

12. The delivery system of claim 11, wherein the outer shell comprises a top cap coupled to a bottom cap.

13. The delivery system of claim 12, wherein the seal is positioned adjacent the top cap and the brush section.

14. The delivery system of claim 11, wherein the bottom cap comprises an O-ring seal for forming a seal between the bottom cap and the base.

15. The delivery system of claim 11, wherein the securing mechanism is a ferrule positioned within the base of the hub for engaging the base and the introducer sheath.

16. A method of delivering at least one medical device into the blood vessel, the method comprising:
securing a hub base to a proximal end of an introducer sheath with a securing mechanism;
securing an outer shell containing at least a seal, a shutter helix section, and a brush section to the hub base and securing a hub cap to the hub base;
inserting the introducer sheath into the blood vessel; and
inserting at least one medical device into a lumen defined by the hub cap, through the outer shell and through the hub base to extend the medical device into the introducer sheath.

17. The method of claim 16, wherein the method further includes inserting a second medical device through the lumen.

18. The method of claim 16, wherein the brush section comprises a plurality of brushes positioned in a helical arrangement, such that upon insertion of the medical device, the plurality of brushes sealingly engage with the medical device.

19. The method of claim 16, wherein the securing mechanism for securing the hub base to the introducer sheath includes a ferrule positioned within the hub base.

20. The method of claim 16, wherein the securing mechanism for securing the hub base to the introducer sheath is a threaded cap for engaging with the proximal end of the introducer sheath and the hub base.
